SEQUENCE LISTING (8 sequences)

SEQ ID NO: 1 - DNA, 29 bp, Artificial Sequence
  Description of Artificial Sequence Synthetic
tcgagtcctgaaggaacggaacagactga

SEQ ID NO: 2 - DNA, 29 bp, Artificial Sequence
  Description of Artificial Sequence Synthetic
tcgatcagtctgttccgttccttcaggac

SEQ ID NO: 3 - DNA, 26 bp, Artificial Sequence
  Description of Artificial Sequence Synthetic
cgtcctgaaggaacggaacagactga

SEQ ID NO: 4 - DNA, 37 bp, Artificial Sequence
  Description of Artificial Sequence Synthetic
tcgatcagtctgttccgtttttccttcaggacgagct

SEQ ID NO: 5 - DNA, 28 bp, Artificial Sequence
  Description of Artificial Sequence Synthetic
tcgagtacatagtacgtgatgttctcaa

SEQ ID NO: 6 - DNA, 28 bp, Artificial Sequence
  Description of Artificial Sequence Synthetic
tcgattgagaacatcacgtactatgtac

SEQ ID NO: 7 - DNA, 31 bp, Artificial Sequence
  Description of Artificial Sequence Synthetic
tcgagtaataggttcttggagtactttacgg

SEQ ID NO: 8 - DNA, 32 bp, Artificial Sequence
  Description of Artificial Sequence Synthetic
tcgaccgtaaagtaactccaagaacctattac

(19) United States
(12) Patent Application Publication
(10) Pub. No.: US 2002/0058290 A1
(43) Pub. Date: May 16, 2002

(54) Cell lines and cell-based assays for identification of androgen receptor modulators

(76) Inventors: Jacek Ostrowski, Jamison, PA (US); Joyce Driscoll, New Hope, PA (US); John Lupisella, New Hope, PA (US); Mark Salvati, Lawrenceville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/885,831

(22) Filed: Jun. 20, 2001

Related U.S. Application Data
(60) Provisional application No. 60/214,392, filed on Jun. 28, 2000.

(51) Int. Cl. G01N 33/567; C12Q 1/66; C12N 5/06

(52) U.S. Cl. 435/7.21; 435/8; 435/354

(57) ABSTRACT

Stable muscle cell lines comprising an androgen receptor and methods of using these cells in functional transactivation assays to assess the efficacy of compounds as androgen receptor modulators in a muscle cell background are provided.

### INTRODUCTION

This application claims the benefit of priority from U.S. Provisional patent application Ser. No. 60/214,392, filed Jun. 28, 2000.

### FIELD OF THE INVENTION

The invention relates to cell lines and methods for using these cell lines in the identification of compounds having biological activity. In particular, the invention relates to muscle cell lines stably transfected with an androgen receptor and reporter gene useful in the identification of compounds which are modulators of the androgen receptor.

### BACKGROUND OF THE INVENTION

The androgen receptor (AR) is a member of the steroid nuclear-receptor superfamily of ligand-dependent transcription factors and is widely distributed among reproductive and nonreproductive tissues, including the prostate and seminal vesicles, male and female genitalia, skin, testis, ovary, cartilage, sebaceous glands, hair follicles, sweat glands, cardiac muscle, skeletal and smooth muscle, gastrointestinal vesicular cells, thyroid follicular cells, adrenal cortex, liver, pineal, and numerous brain cortical and subcortical regions, including spinal motor neurons (Negro-Vilar, A. JCE&M 1999 54(10):3459-62). As with the other members of the steroid receptor family, AR has several functional domains including a DNA binding domain (DBD), and a 261 residue ligand-binding domain (LBD) (Mw=30,245 Da) that contains the androgen binding site, and is responsible for switching on the androgen function. The cDNA and amino acid sequences of human and rat androgen receptors have been described (Proc. Natl. Acad. Sci. U.S.A. 1988 85: 7211- 7215).

AR is an important target in multiple areas of drug discovery and patient therapy. In Oncology, for example, inhibitors (antagonists or partial antagonists) of the androgen receptor function are useful for the treatment of androgen dependent prostate cancer while agonists or partial agonists of the AR are applicable to the treatment of breast cancer. For metabolic and endocrine diseases disorders, for example, agonists or partial agonists of the androgen receptor function are useful for the treatment of age-related diseases and conditions of cachexia in several disease states including, but not limited to, AIDS. Functional AR has also been identified in various bone cells and androgen administration has beneficial effects on skeletal development and maintenance in men and women.

Progress of androgen therapy has been limited by the inability to separate desirable androgenic activities from undesirable or dose limiting side effects. However, recent advances in the development of selective estrogen receptor modulators (SERMS) with a great degree of tissue selectivity in targeting the estrogen receptor while eliminating undesired side effects has resulted in the suggestion of SARMs, selective androgen receptor modulators (Negro-Vilar, A. JCE&M 1999 54(10):3459-62; Reid et al. Investigational New Drugs 1999 17:271-284).

General assays and methods for detecting the transcriptional activity of an intracellular receptor when exposed to a known ligand or unknown compound have been described. For example, U.S. Pat. No. 5,071,773 describes an assay for identifying hormone intracellular receptors, ligands for these receptors and proteins capable of transcriptionally activating the hormone intracellular receptors. The assays involve use of a cell containing DNA encoding a hormone response element such as a promoter linked to an operative reporter gene and DNA encoding the intracellular receptor protein. When the cell is exposed to the hormone or a ligand, a hormone intracellular receptor complex forms and is delivered to an appropriate DNA binding region, thereby activating the hormone response element, which in turn leads to expression of the product encoded by the reporter gene. Activation of the reporter gene is detected in accordance with known procedures for detection of the reporter gene.

U.S. Pat. No. 6,017,924 discloses non-steroidal compounds characterized as high affinity, high specificity agonists, partial agonists (i.e. partial activators and/or tissue-specific activators) and antagonists for androgen receptors based upon a “cis-trans” or “co-transfection” assay. Non-steroidal compounds characterized as high affinity, high specificity agonists, partial agonists (i.e. partial activators and/or tissue-specific activators) and antagonists for androgen receptors via the “cis-trans” or “co-transfection” assay are also described in WO 01/16108, WO 01/16133, and WO 01/16139. This co-transfection assay (Evans et al. Science 1988 240:889-95) is suggested to provide a method for identifying functional agonists and partial agonists which mimic, or antagonists which inhibit, the effect of native hormones, and quantifying their activity for responsive intracellular receptor proteins. In this assay, CV-1 cells (African green monkey kidney fibroblasts) are transiently transfected with the plasmid pRShAR containing the human AR under the constitutive control of the SV40 promoter and a reporter plasmid MTV-LUC containing the cDNA of firefly luciferase under the control of a mouse mammary tumor virus (MTV) long terminal repeat.

The plasmid pRS-β-Gal, coding for constitutive expression of E. coli β-galactosidase, is included as an internal control for evaluation of transfection efficiency and compound toxicity.

Hydroxyflutamide, a known AR antagonist in most tissues, has also been suggested to function as a selective AR modulator (SARM) for effects on IL-6 production by osteoblasts (Hofbauer et al. J. Bone Miner. Res. 1999 14:1330-1337). Selectivity of hydroxyflutamide was assessed by evaluating the proliferation and differentiation of a human fetal osteoblast cell line (HFOB/AR-6) that expresses a mature osteoblast phenotype and a physiological number of androgen receptors in the presence of this compound.

Hydroxyflutamide and Casodex, both known to be full AR antagonists in most tissues, have also been shown, in AR-transfected PC3 cells, to activate MAP kinases Erk-1 and Erk-2 in an AR dependent fashion similar to dihydrotestosterone (DHT; Peterziel et. al. Oncogene 18, 6322-6329 (1999)).

The compound LGD2226, a non-steroidal AR agonist, has also been characterized as a selective androgen receptor modulator for use in the treatment of androgen- related diseases such as osteoporosis, male hormone replacement, male and female sexual dysfunction and cachexia based upon its activity in the CV-1 assay described supra (SCRIP - World Pharmaceutical New FILED May 12, 2000; WO 01/16108; WO 01/16133; and WO 01/16139).

U.S. Pat. No. 5,952,488 describes a bioassay for androgenic materials in cell culture wherein HeLa cells or PC-3 cells are transiently transfected or stably integrated with a DNA sequence cloned from the probasin (PB) gene promoter region coupled to a CAT reporter gene.

U.S. Pat. No. 5,506,102 describes methods and assays useful in screening compounds for potential antagonists of steroid intracellular receptor mediated transcription wherein cells are transfected with a first vector encoding the intracellular receptor, a second vector encoding the PR-A isoform of human progesterone receptor and a third vector encoding a reporter gene.

Applicants have now developed for the first time cell lines and assays in which an AR and reporter have been stably transfected into muscle tissue cells.

### SUMMARY OF THE INVENTION

An object of the present invention is to provide muscle cell lines comprising a mammalian androgen receptor stably introduced into said muscle cells. These cell lines are useful in functional transactivation assays to assess the efficacy of compounds as androgen receptor modulators in a muscle cell background.

Another object of the present invention is to provide functional transactivation assays for use in assessing the efficacy of compounds as androgen receptor modulators in a muscle cell background via these stable C2C12 mouse skeletal muscle cell lines comprising the mammalian androgen receptor.

Yet another object of the present invention is to provide androgen receptor modulators, and in particular selective androgen receptor modulators, identified via functional transactivation assays with stable C2C12 mouse skeletal muscle cell lines comprising a mammalian androgen receptor.

### DETAILED DESCRIPTION OF THE INVENTION

The present invention relates to muscle cell lines stably introduced with a mammalian androgen receptor and reporter gene.

Various muscle cells can be used in the present invention. In a preferred embodiment, the muscle cell line comprises stable C2C12 mouse skeletal muscle cells. However, other exemplary muscle cells useful in the present invention include, but are not limited to, mouse G-7, G-8, P19 and Sol8 cells, rat H9c2(2-1), L6 and L8 cells, and human SJRH30(RMS13) cells.

The muscle cell lines of the present invention further comprise a mammalian androgen receptor which is stably introduced into the muscle cells. Androgen receptors useful in the present invention have been isolated from various mammalian species. These receptors and their sequences have been described in detail in the prior art. For example, see U.S. Pat. No. 5,614,620. In addition, rat androgen receptors are set forth in Genbank Accession No. M23264 and J05454, as well as by Chang et al. (Science 1988 240(4850):324-326). Mouse androgen receptors are set forth in Genbank Accession No. M37890 and by Gaspar et al. (Proc. Natl Acad. Sci. U.S.A. 1991 88:8606-8610) and He et al. (Biochem. Biophys. Res. Commun. 1990 171(2):697- 704). A guinea pig androgen receptor has also been described by He et al. (Biochem. Biophys. Res. Commun. 1990 171(2):697-704). He et al. (Biochem. Biophys. Res. Commun. 1990 171(2):697-704) also describes a dog androgen receptor as does Genbank Accession No. AF197950. A hamster androgen receptor is described by Shiba et al. (J. Dermatol. Sci. 2001 26(3):163-8. In addition, human androgen receptors are set forth in Genbank Accession No. M34233 and by Trapman et al. (Biochem. Biophys. Res. Commun. 1988 153(1):241-248) and Tilley et al. (Proc. Natl Acad. Sci. U.S.A. 1989 86(l):327-331). In a preferred embodiment, the cell lines of the present invention comprise a rat androgen receptor.

The cell lines of the present invention are useful in assessing the activity of compounds as androgen receptor modulators in a muscle cell background.

In one embodiment, the cell line comprises stable

2C12 mouse skeletal cells containing a full length rat androgen receptor such as that set forth in GenBank Accession No. M23264. This cell line is referred to herein as Stable 1 .

To generate the Stable 1 cell line containing the full length rat androgen receptor (rAR), the C2C12 mouse skeletal cell line (Yaffe D. and Saxel, O. Nature 1977 270:725-727) was transfected with a plasmid, pIRESneo/rAR, encoding a bicistronic message containing a full length rAR and the neomycin resistance gene (Jackson et al. Trends Biochem. Sci. 1990 15:477-483; Jang et al. J. Virol. 1988 62:2636-2643). Specifically, 50 μg pIRESneo/rAR were transfected into C2C12 cells using LipofectAmine Plus™ reagent (Gibco BRL) with 250 μl plus reagent and 375 μl lipofectamine reagent in 10 milliliters optiMEM media (Gibco BRL) in accordance with the manufacturer's instructions. Cells (0.75×10⁵) in 10 milliliters growth media (Dulbecco's modified Eagle medium (DMEM) high glucose supplemented with 10% FBS, 1X sodium pyruvate and 0.5X antibiotic-antimycotic (all from Gibco BRL)), referred to hereinafter as Stable 1 growth media, were plated onto each of five 10-cm culture plates. The following day, the media was removed from each dish and replaced with 4.5 milliliters optiMEM. Two milliliters of the transfection mixture were then added to each dish. After a three hour incubation, the transfection media was removed and replaced with 6.5 milliliters of growth media. The cells were allowed to grow for 24 hours in non-selection media. To select for individual cells stably transfected with neo/rAR, the cells were split 1:15 into Stable 1 growth media supplemented with 800 μg/ml G418 and allowed to propagate as separate clonal cell lines. After fourteen days, a total of 80 resistant clones were isolated. Clones exhibiting normal growth characteristics were transiently transfected with the enhancer/promoter/reporter construct, pGL3/2X DR-1/luciferase. Stable 1 cells are identified as clones showing a significant increase in luciferase activity, as measured via the Steady-Glo™ Luciferase Assay System (Promega), upon addition of 0.1 μM dihydrotestosterone (DHT). In a preferred embodiment, the Stable 1 cell line exhibits approximately a 12-fold increase or greater in luciferase activity upon addition of the DHT.

Stable 1 cells of the present invention comprising a stable C2C12 mouse skeletal cell line containing a full length rat androgen receptor were sent for deposit on Jun. 12, 2001 to the American Type Culture Collection (ATCC), 10801 University Boulevard, Manassas, VA USA 20110-2209. Twenty-five vials of Stable 1 cells, with an approximate activity of 30,000 specific relative luminescence units (RLUs) in the presence of 100 nM DHT in the transactivation assay described infra, were shipped to the ATCC. The ATCC Deposit Number for the Stable 1 cell line is XXX.

In another embodiment, the stable C2C12 mouse skeletal cell line contains a full length rat androgen receptor plus an enhancer/promoter/reporter construct. This cell line is referred to herein as Stable 2.

Various enhancer/promoter constructs can be used in construction of the Stable 2 cell line. In a preferred embodiment, the enhancer comprises an androgen response element (ARE). Exemplary AREs used in these constructs include, but are not limited to, C3-1, PB-ARE, and DR1. C3-1 is a consensus ARE/GRE (glucocorticoid receptor response element) isolated from the C3 subunit promoter of the gene for rat prostatic binding protein. 2×C3, containing two C3-1 elements, comprises a consensus enhancer sequence for AR and GR (Claessens et al. J. Biol. Chem. 1996 271:19013-19016). PB-ARE is an androgen receptor specific response element isolated from the promoter of the rat probasin gene (Claessens et al.

J. Biol. Chem. 1996 271:19013-19016). The DR1 response element is also androgen receptor specific, however, it was derived synthetically from a pool of degenerate oligonucleotides containing a consensus ARE/GRE using a random sequence selection and amplification method. 1X DR- 1, containing 1 DR-1 element, and 2X DR-1, containing two DR-1 elements, have both been reported as specific for AR (Zhou et al. J. Biol. Chem. 1997 272:8227-8235). Each DR1 element consists of two AR core binding sites oriented as an overlapping direct repeat (Zhou et al.. J. Biol. Chem. 1997 272:8227-8235).

Various promoters can also be used in these constructs. Exemplary promoters include, but are not limited to, SV40, CMV, beta-globin, and HSVtk. However, as will be understood by those of skill in the art upon reading this disclosure, other promoters useful in the present invention can be routinely selected.

In a preferred embodiment, the enhancer/promoter construct of the Stable 2 cell line comprises pGL3/2X DR-1 which carries the stronger SV40 promoter. 2XDR-1 was reported to be an AR specific response element in CV-1 cells (Zhou et. al. J. Biol. Chem. 1997 272:8227-8235). It was developed by random mutagenesis of an AR/GR consensus enhancer sequence. Experiments described in detail in Example 2 showed 2X DR-1 to exhibit better stimulation and selectivity upon addition of DHT as compared to enhancer/promoter constructs comprising AREs C3 , 1X DR-1, as well as PB-ARE. However, alternative enhancer/promoter constructs which can be used to construct the Stable 2 cell line of the present invention include, but are not limited to, pGL3/2XC3, pGL3XDR-1, pGL3/PB-ARE, HSVtk/2XC3, HSVtk/1XDR-1, HSVtk/2XDR-1 and HSVtk/PB-ARE. Construction of these enhancer/promoter constructs is described in detail herein in Example 1.

Various reporter genes can also be used in the construct. Examples include, but are not limited to, luciferase, beta-galactosidase, secretory alkaline phosphatase, beta-lactamase, numerous green fluorescence proteins, and chloramphenicol acetyltransferase. In a preferred embodiment, the reporter gene is luciferase.

To generate the Stable 2 cell line containing the full length rat androgen receptor plus the enhancer/promoter/reporter construct, pGL3/2X DR- 1/luciferase, cells of the Stable 1 cell line were cotransfected with a plasmid containing the enhancer/promoter/reporter construct and a plasmid conferring resistance to hygromycin B (pcDNA3.1-/Hygro, Invitrogen, Carlsbad, CA). Specifically, 60 μg pGL3/2XDR-1 luciferase and 15 μg pCDNA3.1-/Hygro were transfected into Stable 1 cells using LipofectAMINE Plus™ reagent (Gibco BRL) with 300 μl plus reagent and 450 μl lipofectamine reagent in 12 milliliters optiMEM media (GibcoBRL) in accordance with the manufacturer's instructions. Cells (6.0×10⁵) in 10 milliliters of Stable 1 growth media supplemented with 800 μg/ml G418 were plated onto each of six 10-cm culture plates. The following day, the media was removed from each dish and replaced with 4.5 milliliters optiMEM. Two milliliters of the transfection mixture were then added to each dish. After a three hour incubation, the transfection media was removed and replaced with 6.5 milliliters of Stable 1 growth media. The cells were allowed to grow overnight and then split 1:18 and 1:24 into Stable 1 growth media supplemented with 800 μg hygromycin B to select for individual cells stably transfected with the enhancer/promoter/reporter construct as well as hygromycin B resistance and then allowed to propagate as separate clonal lines. After fourteen days, resistant clones were isolated and clones exhibiting normal growth characteristics were tested for luciferase activity in the presence of 0.1 μM DHT. Clones with activities ranging from 3- to 12-fold increase over background were expanded.

Further characterization using standard reference compounds DHT, fluoxymestrone, oxandrolone and medroxyprogesterone acetate was performed on several of these clones. In a preferred embodiment, the Stable 2 cell line exhibits a 12X increase or greater over background in luciferase activity upon addition of the DHT with an EC₅₀ in the sub-nanomolar range. The expected activity was exhibited when the Stable 2 cells were exposed to the other reference compounds.

Stable 2 cells of the present invention comprising a stable C2C12 mouse skeletal cell line containing a full length rat androgen receptor and a stably transfected pGL3/2X DR-1/luciferase reporter were sent for deposit on Jun. 12, 2001 to the American Type Culture Collection 15 (ATCC), 10801 University Boulevard, Manassas, Va. U.S.A. 20110- 2209. Twenty-five vials of Stable 2 cells, with an approximate activity of 30,000 specific relative luminescence units (RLUs) in the presence of 100 nM DHT in the transactivation assay described infra, were shipped to the ATCC. The ATCC Deposit Number for the Stable 2 cell line is XXX.

The present invention also relates to functional transactivation assays developed to assess the activity of compounds as androgen receptor modulators in a muscle cell background via detection of expression of a reporter gene. By “modulator”, for purposes of the present invention, it is meant to be inclusive of agonists, partial agonists, antagonists, and/or partial antagonists of AR.

In these assays, efficacy of a compound as an 30 androgen receptor agonist or partial agonist is assessed by contacting either Stable 1 cells transiently transfected with an androgen response element, a promoter and a reporter gene or Stable 2 cells with a compound and detecting reporter gene expression in the cells in the presence of the compound. An increase in reporter gene expression in the cells in the presence of the compound, as compared to control cells not contacted with or exposed to the compound, is indicative of the compound being an androgen receptor agonist or partial agonist in muscle cells.

Efficacy of a compound as an androgen receptor antagonist or partial antagonist is assessed by a competition assay wherein the ability of a compound to prevent the induction of expression of a reporter gene by DHT in Stable 1 or Stable 2 cells is determined. In a preferred embodiment, approximately 1 nM DHT is used in the assay to induce expression of the reporter gene. A decrease in reporter gene expression in the presence of the compound as compared to control cells exposed to DHT, but not contacted with the compound, is indicative of the compound being an androgen receptor antagonist or partial antagonist in muscle cells. These assays can be used to determine the concentration at which the compound inhibits DHT induction by 50%, also referred to as the IC₅₀. More specifically, a first assay of the present invention, referred to herein as Androgen Receptor Transactivation Assay (ARTA) Stable 1, uses the Stable 1 cell line, which stably expresses the full length rat androgen receptor but requires the transient transfection of an enhancer/promoter/reporter construct. In this assay, Stable 1 cells are plated, preferably in a 96 well format, at approximately 5,000 to 10,000 cells/well, preferably 6,000 cells/well, in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1X MEM Na Pyruvate (Gibco BRL, Cat. No.: 11360-070), 0.5X Antibiotic- Antimycotic, and 800 μg/ml Geneticin (Gibco BRL, Cat. No.: 10131-035). Once the cells have adhered and acclimated and reached optimal confluency for transfection, approximately twenty-four hours after plating, the cells are transfected with an enhancer/promoter/reporter construct such as pGL3/2XDR-1/luciferase.

Preferably, the transfection is performed using LipofectAMINE Plus™ Reagent (Gibco BRL, Cat. No.: 10964- 013). In this preferred embodiment, pGL3/2XDR-1/luciferase DNA (approximately 5 ng/well) and a carrier, such as Salmon Sperm DNA (50 ng/well) or a generic plasmid DNA, are diluted with 5 μl/well Opti-MEM media (Gibco BRL, Cat. No.: 31985-070). To this, 0.5 μl/well Plus reagent is added. This mixture is incubated for 15 minutes at room temperature. In a separate vessel, 0.385 μl/well LipofectAMINE reagent is diluted with 5 μl/well Opti-MEM. The DNA mixture is then combined with the LipofectAMINE mixture and incubated for an additional 15 minutes at room temperature. During this time, the media from the cells is removed and replaced with 60 μl/well of Opti-MEM. To this is added 10 μl/well of the DNA/LipofectAMINE transfection mixture. The cells are incubated for 4 hours. The transfection mixture is removed from the cells and replaced with 90 μl of the high glucose DMEM described supra.

Other transfection methods which can be used in the present invention include, but are not limited to, DEAE- dextran, calcium phosphate, direct microinjection, electroporation, and biolistic particle delivery.

Compounds to be tested for activity in this assay are then placed in each well. In a preferred embodiment, 10 μl of appropriate compound dilution is placed in each well. It is preferred that a range of concentrations of compound, i.e. from about 0.001 nM to 3000 nM, be tested. It is also preferred that initial dilutions of compounds be made in dimethylsulfoxide or ethanol and that subsequent dilutions be made in assay media. Twenty-four hours later activity of the compound is detected via a detection system such as the Steady-Glo™ Luciferase Assay System (Promega, Cat. No.: E2520), or via other luciferin substrates (Tropix or Packard Biosciences) according to the manufacturers' instructions.

A second assay of the present invention, referred to herein as ARTA Stable 2, uses the Stable 2 cell line, derived from Stable 1 which stably expresses both rat androgen receptor and an ARE enhancer/promoter/reporter construct. The enhancer/promoter/reporter construct used in this system preferably comprises pGL3/2XDR-1/luciferase.

In the ARTA Stable 2 assay, Stable 2 cells are plated, preferably in 96 well format, at approximately 5,000 to 10,000 cells/well, preferably 6,000 cells/well, in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1X MEM Na Pyruvate (Gibco BRL, Cat. No.: 11360-070), 0.5X Antibiotic-Antimycotic, 800 μg/ml Geneticin (Gibco BRL, Cat. No.: 10131-035) and 800 μg/ml Hygromycin B (Gibco BRL, Cat. No.: 10687-010). Approximately 24 hours later, the media on the cells is removed and replaced with 90 μl fresh assay media.

Compounds to be tested for activity in this assay are then placed in each well. In a preferred embodiment, a 10 μl aliquot of compound at a concentration ranging from about 0.001 nM to 3000 nM, is placed in each well. It is preferred that initial dilutions of a compound be made in dimethyl sulfoxide or ethanol and subsequent dilutions be made in assay media. After 24 hours, activity is detected via the Steady-Glo™ Luciferase Assay System (Promega, Cat. No.: E2520) or via other luciferin substrates (Tropix or Packard Biosciences) according to the manufacturers' instructions.

An agonist or partial agonist, for purposes of the present invention, is defined as any compound that achieves 50% of the maximal activity of DHT at a concentration less than or equal to 3000 nM (3 μM) in the transactivation assay of the present invention.

An antagonist or partial antagonist, for purposes of the present invention, is defined as any compound that is able to inhibit by 50% the maximal activity of 1 nM DHT at a concentration less than or equal to 3000 nM in the transactivation assay of the present invention.

The assays of the present invention are particularly useful in identifying specific or selective androgen receptor modulators or SARMs. By “SARM” it is meant an androgen receptor modulator exhibiting a difference-in-kind of the modulation effected in one type of tissue, i.e. tumors, containing the androgen receptor relative to the modulation effected in other tissues, i.e. nontumor tissues, containing the androgen receptor.

In this embodiment, the agonist or antagonist activity of a potential SARM is measured in an assay of the present invention to ascertain activity of the compound in a muscle cell background. Activity of the potential SARM can also be measured in other nontumor cells lines such as primary rat prostate epithelial and stromal cells, primary guinea pig smooth muscle cells, primary smooth-muscle cells from immature (I-PSMC) or adult (A-PSMC) rat penis, primary rabbit smooth muscle cell line, prostatic smooth muscle cell line PS-1, prostatic smooth muscle cell line PSMC1, mouse bone cell cultures and osteoblasts cells and primary rat seminal vesicle lines SVC-1 and SCV-2. Such cell lines are described in the following exemplary references and the references contained therein: Nemeth et. al. J. Andrology 19, 718-724 (1998), Zhuang et. al. J. Steroid Biochem. Mol. Biol. 41, 693-696 (1992), Zhang et. al. Prostate 30, 117-129 (1997), Ricciardelli et. al. J. Endocrinol. 140, 373-383 (1994), Gonzalez-Cadavid et. al. Mol. Cell. Endocrinol. 90, 219-229 (1993), Sadeghi-Nejad et. al. Int. J. Impotence Res. 10, 165-169 (1998), Gerdes et. al. Endocrinology 139, 3569-3577 (1998), Sarah et. al. J. Cell. Physiol. 185, 416-424 (2000), Chen et. al., FEBS Letters 491, 91-93 (2001) and Tajana et. al. EMBO J. 3, 637-644 (1984).various methods for identifying SARMs having antagonist activity against hormone-dependent tumors while exhibiting no activity, or more preferably agonist activity against other nontumor tissues containing the androgen receptor can be used.

The agonist or antagonist activity of the potential SARM is then also ascertained in hormone-dependent tumors via screening for inhibition of growth in hormone-dependent tumor cell lines. Examples of hormone-dependent tumor cell lines which can be used for screening potential SARMs include, but are not limited to, human breast tumor cell line MDA MB453, human breast tumor cell line ZR-75-1, murine breast line Shionogi, rat prostate adenocarcinoma line Dunning R-3327, human prostate tumor cell line MDA PCa 2a and PCa 2b, human prostate cell line LNCap, human prostate tumor cell line CWR22, human prostate tumor cell line LuCaP 35 and LuCaP 23.12, human prostate cell line LAPC-4 and LAPC-9, human prostate tumor cell line PC-295, human prostate tumor cell line PC-310, and human osteosarcoma cell line MG-63. These experimental human and murine prostate and breast cell lines are well accepted by those of skill in the art as indicative of the pharmacology of human hormone-dependent tumors, such as prostate cancer. Examples of the relationship of such models to the human disease state can be found in, but are not limited to, the following references and the references contained therein, Jacques et. al. Endocrinology 140, 416-421 (1999); Yeap et. al. Endocrinology 140, 3282-3291 (1999), Sharma et. al. Oncogene 18, 5349-5355 (1999), Isaacs, J. T. Urol. Oncol. 2, 115-116 (1996), Bentei et. al. In Vitro Cell Dev. Biol. 35, 655-662 (1999), Suzuki et. al. J. Steroid Biochem. Mol. Biol. 37, 559-567 (1990), Peehl, D. M. Urol. Oncol. 2, 100-102 (1996), Wytske et. al. Urol. Oncol. 2, 122-125 (1996), Leland, C. W. K. Urol. Oncol. 2, 126-128 (1996), Buhler et. al. The Prostate 43, 63-70 (2000), Navone et. al. Clin. Cancer Res. 6, 1190-1197 (2000), Etreby et. al. The Prostate 42, 99-106 (2000), Jongsma et. al. Cancer Res. 60, 741-748 (2000), Jongsma et. al. Amer. J. Path. 154, 543-551 (1999), Ye et. al. Clin. Cancer Res. 5, 2171-2177 (1999), Navone et. al. Clin. Cancer Res. 3, 2493-2500 (1997), Klein et. al. Nature Medicine 3, 402-408 (1997), Chen et. al. Cancer Res. 58, 2777-2783 (1998), and Craft et. al. Cancer Res. 59, 5030-5036 (1999).

Preferred SARMs identified via assays of the present invention are those exhibiting antagonist activity in tumors versus agonist activity in other, nonmalignant tissues containing the androgen receptor. SARMs identified in accordance with these assays as agonists of androgen receptors in muscle tissue are useful in inhibiting muscle wasting and cachexia oftentimes observed in patients suffering from cancer or AIDS.

The following nonlimiting examples are provided to further illustrate the present invention.

### EXAMPLES

### Example 1

### Construction of Plasmids

Androgen Receptor Plasmid pIRESneo/rAR

The rat androgen receptor (GenBank Accession No. M23264) was subcloned as a NotI fragment into the NotI site of pIRESneo (Clontech Laboratories, Palo Alto, CA).

ARE/Luciferase Reporter Plasmids

A series of luciferase reporter constructs containing known androgen receptor response elements (AREs), C3, DR-1 and PB-ARE were prepared in the pGL3-Promoter vector (Promega Corporation, Madison, WI).

pGL3/1XDR-1/Luci ferase

Equimolar amounts of the complementary oligonucleotide DR-1(F) and DR-1(R) were annealed and then ligated into the XhoI digested pGL3-Promoter plasmid (Promega Corporation). The oligonucleotide DR-1(F) has the sequence:

5′-TCGAGTCCTGAAGGAACGGAACAGACTGA-3′ (SEQ ID NO:1). The oligonucleotide DR-1(R) has the sequence:

5′-TCGATCAGTCTGTTCCGTTCCTTCAGGAC-3′ (SEQ ID NO:2).

pGL3/2XDR-1 Luciferase

A second DR-1 response element was inserted upstream of the existing DR-1 element in pGL3/1XDR-1/Luciferase by annealing equimolar amounts of the complementary oligonucleotide 1XDR-1 (F) and 1XDR-1 (R) and then ligating into SacI/XhoI digested pGL3/1XDR-1/Luciferase plasmid. The oligonucleotide 1XDR-1 (F) has the sequence:

5′-CGTCCTGAAGGAACGGAACAGACTGA-3′ (SEQ ID NO:3). The oligonucleotide 1XDR-1 (R) has the sequence:

5′-TCGATCAGTCTGTTCCGTTTTTCCTTCAGGACGAGCT-3′ (SEQ ID NO:4).

pGL3/2XC3-1/Luciferase

Equimolar amounts of the complementary oligonucleotides C3-1(F) and C3-1(R) were annealed and then ligated to each other. Gel purified dimers were then ligated into the XhoI digested pGL3-Promoter plasmid (Promega Corporation). The oligonucleotide C3-1(F) has the sequence:

5′-TCGAGTACATAGTACGTGATGTTCTCAA-3′ (SEQ ID NO:5). The oligonucleotide C3-1(R) has the sequence:

5′-TCGATTGAGAACATCACGTACTATGTAC-3′ (SEQ ID NO:6).

pGL3/2XPB-ARE/Luciferase

Equimolar amounts of the complementary oligonucleotide PB-ARE-2F and PB-ARE-2R were annealed and then ligated to each other. Gel purified dimers were then ligated into the XhoI digested pGL3-Promoter plasmid (Promega Corporation). The oligonucleotide sequence of PB- ARE-2F has the sequence:

5′-TCGAGTAATAGGTTCTTGGAGTACTTTACGG-3′ (SEQ ID NO:7). The oligonucleotide sequence of PB-ARE-2R has the sequence:

5′-TCGACCGTAAAGTAACTCCAAGAACCTATTAC-3′ (SEQ ID NO:8).

PGL3/HSVtk

This vector was prepared by replacing the SV40 promoter in pGL3-Promoter plasmid (Promega Corporation) with the HSVtk (Herpes Simplex Virus Thymidine Kinase) promoter from PRL-TK (Promega Corporation). Both promoters are contained within Bgl II/Hind III fragments and were easily exchanged by ligating the HSVtk fragment from PRL-TK into the Bgl II/Hind II digested pGL3-Promoter vector.

pGL3/HSVtk/1XDR-1/Luciferase, pGL3/HSVtk/2XDR-1/Luciferase,

pGL3/HSVtk/2XC3-1/Luciferase and pGL3/HSVtk/2XPB-ARE/Luciferase

These constructs containing the HSVtk promoter in place of the SV40 promoter were prepared by replacing the SV40 promoter in the respective parent plasmid with the HSVtk promoter from pRL-TK as described for pGL3/HSVtk.

### Example 2

### Selection of Enhancer/Reporter Construct

Two sets of vectors with luciferase as a reporter were constructed and tested in the C2C12 mouse skeletal muscle cell line. The first variant of the luciferase reporter construct carried a strong promoter, in this specific example the SV40 promoter. The second variant of the luciferase reporter construct carried a basal promoter, in this specific example the HSVtk promoter. To select the most effective androgen response element (ARE) to drive expression of the luciferase gene, both SV40 and HSVtk promoters were coupled to four different AREs, C3, DR-1 (lX and 2X) and PB-ARE. The C3 enhancer is a strong androgen dependent regulatory element with a crossover activity with Glucocorticoid Receptor (GR). Both DR-1 and PB-ARE are considered to be specific androgen response elements.

A transient transactivation experiment was performed in which CMVrAR was cotransfected with the aforementioned enhancer/promoter/reporter construct (10:1 receptor to enhancer/promoter/reporter) in C2C12 cells using LipofectAMINE Plus™ reagent (GibcoBRL) according to the manufacturer's instructions was used to compare the activities of the enhancer/promoter/reporter constructs. Specifically, 10,000 cells/well were plated in growth media (Dulbecco's modified Eagle Medium (DMEM) high glucose supplemented with 10% FBS, 1X sodium pyruvate and 0.5X antibiotic-antimycotic (all from GibcoBRL)). The next day, the media was removed and replaced with optiMEM media (Gibco BRL). The transfection mixture was prepared so that 10 μl added to each well resulted in 0.05 μg/well receptor, 0.005 μg/well enhancer/promoter/reporter construct and 0.385 μl/well lipofectamine. After three hours of incubation, the transfection mixture was removed and replaced with growth media which had been made using charcoal/dextran FBS (Hyclone, Logan UT). The method of detection used was the Steady-Glo™ Luciferase Assay System (Promega Corporation) with counting performed on a Packard TopCount (Packard Instrument Co. Downers Grove, IL).

Results showed that C3, DR-1, and PB-ARE/HSVtk- luciferase reporter constructs had a lower background signal as compared to C3, DR-1, and PB-ARE/SV40 luciferase reporters. Addition of 1 μM testosterone gave a 3.5 fold increase over background with C3/HSVtk/luciferase, and a 2.0-, 2.0- and 6.5-fold increase with 2XPB-ARE-HSVtk/luciferase, IXDR-1/HSVtk/luciferase and 2XDR-1/HSVtk/luciferase, respectively. The constructs with the greatest fold window of stimulation, C3 and DR-1/HSVtk/luciferase, both showed a minimum 100-fold selectivity of testosterone over dexamethasone when tested in dose response experiments. Therefore, for reasons of fold window of stimulation and selectivity, the 2XDR-1 construct was selected. Although in the transiently transfected receptor system, the HSVtk promoter seemed preferable due to the lower background, when tested in the Stable 1 cell line the signal greatly diminished. Therefore, the construct used in the production of the Stable 2 cell line was pGL3/2XDR-1/luciferase which carries the stronger SV40 promoter.

What is claimed is:

1. A stable muscle cell line comprising muscle cells and a mammalian androgen receptor stably introduced into said muscle cells.

2. The stable muscle cell line of claim 1 wherein the muscle cells comprise C2C12 mouse skeletal muscle cells.

3. The stable muscle cell line of claim 1 wherein the mammalian androgen receptor comprises a rat androgen receptor.

4. The stable muscle cell line of claim 1 further comprising a stably transfected enhancer/promoter/reporter construct.

5. The stable muscle cell line of claim 4 wherein the enhancer comprises an androgen response element.

6. The stable muscle cell line of claim 5 wherein the enhancer comprises C3-1, DR-1 or PB-ARE.

7. The stable muscle cell line of claim 4 wherein the promoter comprises SV40.

8. The stable muscle cell line of claim 4 wherein the promoter comprises HSVtk.

9. The stable muscle cell line of claim 4 wherein the reporter gene comprises luciferase.

10. The stable muscle cell line of claim 4 wherein the enhancer/promoter/reporter construct comprises pGL3/2XDR-1/luciferase.

11. A stable muscle cell line comprising ATCC Deposit XXX.

12. A stable muscle cell line comprising ATCC Deposit XXX.

13. A functional transactivation assay for assessing efficacy of a compound as an androgen receptor agonist or partial agonist comprising:
(a) transiently transfecting the cell line of claim 1 with a plasmid containing an androgen response element, a promoter and a reporter gene;
(b) contacting the transiently transfected cell line with a compound; and
(c) detecting reporter gene expression in the transiently transfected cell line, wherein an increase in reporter gene expression in the transiently transfected cell line in the presence of the compound is indicative of the compound being an androgen receptor agonist or partial agonist.

14. An androgen receptor modulator comprising a compound identified in accordance with the method of claim 13.

15. A functional transactivation assay for assessing efficacy of a compound as an androgen receptor antagonist or partial antagonist comprising:
(a) transiently transfecting the cell line of claim 1 with a plasmid containing an androgen response element, a promoter and a reporter gene;
(b) contacting the transiently transfected cell line with a compound and dihydrotestosterone; and
(c) detecting reporter gene expression in the transiently transfected cell line, wherein a decrease in reporter gene expression in the transiently transfected cell line in the presence of the compound and dihydrotestosterone as compared to cells exposed only to dihydrotestosterone is indicative of the compound being an androgen receptor antagonist or partial antagonist.

16. An androgen receptor modulator comprising a compound identified in accordance with the method of claim 15.

17. A functional transactivation assay for assessing efficacy of a compound as an androgen receptor agonist or partial agonist comprising:
(a) contacting the cell line of claim 4 with a compound; and
(b) detecting reporter gene expression in the cell line, wherein an increase in reporter gene expression in the cell line in the presence of the compound is indicative of the compound being an androgen receptor agonist or partial agonist.

18. An androgen receptor modulator comprising a compound identified in accordance with the method of claim 17.

19. A functional transactivation assay for assessing efficacy of a compound as an androgen receptor antagonist or partial antagonist comprising:
(a) contacting the cell line of claim 4 with a compound and dihydrotestosterone; and
(b) detecting reporter gene expression in the cell line, wherein a decrease in reporter gene expression in the presence of the compound and dihydrotestosterone as compared to cells exposed only to dihydrotestosterone is indicative of the compound being an androgen receptor antagonist or partial antagonist.

20. An androgen receptor modulator comprising a compound identified in accordance with the method of claim 19.
